(19) United States
(12) Patent Application Publication
(10) Pub. No.: US 2018/0153840 A1
(43) Pub. Date: Jun. 7, 2018

(54) THE COMPOSITIONS OF GLYCEROL AND /OR NON-TOXIC AMINO ACIDS FOR INHIBITING AND DESTROYING BIOFILM, INCLUDING RELATED METHODS

(71) Applicant: Thomas Benedict Bryan, Merced, CA (US)

(72) Inventor: Thomas Benedict Bryan, Merced, CA (US)

(21) Appl. No.: 15/857,647

(22) Filed: Dec. 29, 2017

Related U.S. Application Data
(63) Continuation-in-part of application No. 15/339,734, filed on Oct. 31, 2016.
Application No. 15/339,734 is a continuation of application No. 14/590,002, filed on Jan. 6, 2015, now Pat. No. 9,480,669.

(51) Int. Cl. A61K 31/198; A61K 31/401; A61K 9/00; A61K 31/4172; A61K 31/405; A61K 31/047; A61K 9/08; A61K 47/10; A61K 47/18

(52) U.S. Cl. CPC A61K 31/198; A61K 31/401; A61K 9/0019; A61K 9/0043; A61K 31/4172; A61K 2300/00; A61K 9/0014; A61K 31/047; A61K 9/08; A61K 47/10; A61K 47/183; A61K 31/405

(57) ABSTRACT

The invention relates to compositions that include non-toxic, non-bonded amino acids, individually or in combination, with or without glycerol and other ingredients, used to prevent and treat disease caused by biofilm producing bacteria, fungi, hybrid, or protozoan microorganisms in animals including humans, and to generally prevent reduce, or destroy biofilms by inhibition of formation and in destruction of said biofilms in various other applications.

### CROSS REFERENCES OF RELATED APPLICATION

This application is a continuation application of U.S. application Ser. No. 15/339,734, filed Oct. 31, 2016, pending, which is a divisional of U.S. Pat. No. 9,480,669, filed Jan. 6, 2015 and issued Nov. 1, 2016, both of which are incorporated herein by this reference in their entireties.

### FIELD OF INVENTION

This invention pertains to the use of certain non-toxic amino acids in order to destroy and inhibit the formation of bacterial and fungal biofilm. The embodiments disclosed herein relate to compositions comprising amino acids, individually and in combination, to treat disease in animals, including humans, and to aid in sterilization.

### BACKGROUND OF THE INVENTION

A biofilm occurs when microbes stick to each other on a surface. These adherent microbial cells are frequently embedded within a self-producing matrix of extracellular polymeric substance. Biofilms are also referred to as slime. The polymeric conglomeration is generally composed of extracellular DNA, proteins and polysaccharides. Initially the biofilm is weak and adhesion is by van der Waals forces. Later, the microbes form cell adhesion structures such as pili in the case of bacteria or hyphae in the case of fungi. Once colonization has begun, the biofilm grows through a combination of cell division and recruitment of extracellular components. They tend to grow on wet surfaces. Microorganisms that form biofilm include: bacteria, fungi, and protists.

Danish pioneers first connected biofilms with human disease and then with antibiotic resistant infections in the late 1970's through the 1980's. They discovered that once these biofilm infections had begun they are difficult to get rid of in the body. The immune system can effectively attack free-floating microbes in the blood but it is less effective at reaching bacteria and fungi within the biofilm reservoir.

According to the Center for Disease Control, 65% of treated bacterial infections develop a biofilm. Biofilms are implicated in chronic infections. Most notable among them is Staphylococcus aureus, especially the methicillin resistant (MRSA) variety. Also, an estimated 13% of intensive care patients have a fungal infection likely originating from a biofilm such as Candida Albicans which is especially troublesome for immuno-depressed patients.

The development of a biofilm may allow for an aggregated cell colony to be increasingly antibiotic resistant and also resistant to the host's defense mechanisms. Microbes from the biofilm can disperse which causes the spread and colonization of new surfaces. The extracellular matrix protects the microorganisms within it and facilitates communication among the microorganisms through biochemical signals. The proximity of cells within the biofilm can facilitate plasmid exchange and thus can enhance antimicrobial resistance. Biofilms have been implicated in such problems as urinary tract infections, endocarditis, cystic fibrosis and infections of medical devices, such as prostheses and heart valves. Invariably, the only recourse for treating prosthetic devices such as mechanical heart valves is to have them replaced. Biofilms are present on the removed tissue of 80% of patients undergoing surgery for chronic sinusitis.

There is also evidence of hybrid colonies of fungal and bacterial cells that can occur in the body that form altered biofilms that have enhanced characteristics that make the hybrid microorganisms more virulent. In one example, people with Crohn's disease have increased amounts of Candida tropicalis fungus, and E. coli and Serratia marcescens bacteria. The combination of these fungal and bacterial cells in the same colony results in the C. tropicalis forming long filaments with E. coli cells fusing to these fungal growths, and S. marcescens forms protein strings that stabilize the biofilm. These hybrids can become very virulent in that they can increase in numbers very rapidly.

Human parasites that cause biofilm include Protozoans, such as Trypanosoma that causes Chagas disease and sleeping sickness, Giardia that causes giardiasis, and Plasmodium that causes malaria may also be treated.

Biofouling in drinking water distribution systems and food processing environments is a common source of pathologic microorganisms.

Treatment of infections that develop biofilms is challenging. While the pathogen itself may be susceptible to an antimicrobial agent, where a biofilm has developed the pathogen may be shielded from the antimicrobial agent. The biofilm matrix acts as a physical barrier that prevents antimicrobial agents from reaching the microbes. Catalases and lactamase in the biofilm can inactivate the antimicrobials before they can reach their target. Low oxygen concentrations in the biofilm also protect the microbes from some antimicrobial agents which require aerobic metabolism to properly function. Additionally, a large portion of the pathogens may be insensitive to the specific antimicrobial agent, as microorganisms in a biofilm typically exist in a dormant state. The dormant microbes are not vulnerable to the antimicrobial agent. Later, these dormant microbes can quickly renew the biofilm.

### SUMMARY OF INVENTION

Disclosed are therapeutic compositions for treating biofilm infections that include a therapeutic amount of one or more non-bonded amino acids, and methods of using the same. The therapeutic composition may be administered to effect the destruction and inhibition of microorganism biofilm. Also disclosed is the ability of glycerol to aid these amino acids in the destruction and inhibition of these microorganisms.

I treated a 66 year old female who developed cellulitis and an open ulcer on her leg. This was treated by oral antibiotics and was also treated at an outpatient wound care center. When the outpatient care was not successful she was admitted to the hospital for intravenous antibiotics. After 3 weeks there was no sign of improvement and the ulcer was enlarging. At this stage the ulcer measured 3 centimeters by 1.5 centimeters in size and there was surrounding redness suggestive of inflammation. The patient was given an intravenous infusion, known as procalamine, consisting of 3% glycerol and 3% amino acids at a rate of 80 cubic centimeters (cc) per hour. At the end of 48 hours there was evidence of healing in the ulcer. During these 48 hours, the patient continued to receive intravenous antibiotics. After 72 hours the patient was discharged home on oral antibiotics. When she was reexamined 3 weeks later, there was no evidence of the ulcer and the surrounding inflammation that was caused by cellulitis had totally cleared. The inventor has found that such ulcers are resistant to antimicrobial (e.g., antibiotic) treatments because the ulcers have developed a bacterial biofilm that provides resistance to the antimicrobial agents.

The composition of this infusion contained the amino acids: Isoleucine 0.21 g per 100 ml, Leucine 0.27 g per 100 ml, Lysine (as Lysine Acetate USP 0.31 g) 0.22 g per 100 ml, Methionine 0.16 g per 100 ml, Phenylalanine 0.17 g per 100 ml, Tryptophan 0.046 g per 100 ml, Valine 0.2 g per 100 ml, Alanine 0.21 g per 100 ml, Arginine 0.29 g per 100 ml, Histidine 0.085 g per 100 ml, Proline 0.34 g per 100 ml, Serine 0.18 g per 100 ml, Glycine 0.42 g per 100 ml, Threonine 0.12 g per 100 ml, Cysteine (as L-cysteine hydrochloride monohydrate less than 0.020 g) less than 0.014 g per 100 ml, plus 3% glycerol as a source of calories.

At my direction, further tests were conducted with the above-mentioned composition for its effect against bacterial and fungal biofilm. The solution was found to be effective against both bacterial and fungal biofilm.

At my direction, in vitro testing of various compositions comprising non-toxic, non-bonded amino acids on bacterial and fungal biofilms was subsequently conducted. Such compositions were found to be effective against both bacterial and fungal biofilm.

At my direction, the effect of glycerol alone on biofilms was tested and was found to have no measurable effect under the experimental conditions. However, it was found that glycerol had either an additive effect on the ability of amino to inhibit and destroy biofilm or have a negative ability on the amino acids that promote biofilm formation.

Moved for Clarity and Compactness.

At my direction, in vitro testing was also done the compositions were also tested for their ability to promote bacterial and fungal biofilm and also to find amino acids that would be neutral in their effect on biofilm.

At my direction, further investigations of the effects on biofilms of non-bonded forms of the non-toxic amino acids, including aspartic acid, cysteine, glutamic acid, beta-alanine, 2-aminoadipic acid, cystathionine, ethanolamine, homocysteine, phosphoethanolamine, phosphoserine, alanine, arginine, asparagine, citrulline, glycine, isoleucine, leucine, lysine, methionine, phenylalanine, ornithine, proline, serine, taurine, threonine, tryptophan, 2-aminobutyric acid, glutamine, histidine, 1-methyl-histidine, 3-methyl-histidine, tyrosine, hydroxyproline, and valine. These amino acids were evaluated for their effect in both the inhibition of formation and in the destruction of bacterial and fungal biofilm. Many of the tested compositions were found to be effective against both bacterial and fungal biofilms. Some of the amino acids were found to promote bacterial or fungal biofilms. Some of the amino acids were found to be neutral and have no effect on bacterial or fungal biofilms.

### SUMMARY OF EMBODIMENTS

Non-toxic, non-bonded amino acids and combinations thereof were examined for their effects on bacterial and fungal biofilms (see Tables C and D). The disclosure relates to pharmaceutical compositions or formulations comprising one or a plurality of non-bonded amino acids or pharmaceutically acceptable salts thereof for treatment of bacterial, fungal, hybrid, and protozoan infections that produce biofilms. The disclosure also relates to methods of treating fungal, bacterial, and protozoan infections by administering the pharmaceutical compositions or formulations disclosed herein. Also provided herein are the compositions, pharmaceutical compositions or formulations for use in the manufacture of a medicament for the treatment of a fungal infection, a bacterial infection, and/or a protozoan infection. The present invention also relates to decontamination solutions that include one or more non-bonded amino acids or salts thereof for treating surfaces, equipment, and other structures.

For example, the compositions and methods described herein may be used to treat fungal infections or bacterial infections caused by antibiotic-resistant bacteria and antifungal agent resistant fungi or other infections caused by an organism that produces a biofilm. Non-exclusive examples of bacterial infections that may produce biofilms and that may be treated using the compositions and methods of the present invention include Streptococcus pneumoniae, Bacillus spp, Listeria monocytogenes, Staphylococcus spp, and lactic acid bacteria, including Lactobacillus plantarum and Lactococcus lactis, and/or Streptococcus mutans. These are non-limited examples of bacterial infections, and the present compositions and methods of the present invention may be used to treat infections caused by various bacteria, fungi, and protozoans.

The non-toxic, non-bonded amino acids included in the pharmaceutical compositions and formulations of the present invention are single amino acids or pharmaceutically acceptable salts thereof with a free amino or carboxy group not covalently bound to another molecule or chemical substance. In some embodiments, the composition, pharmaceutical composition or decontamination solution of the disclosure comprises at least one amino acid in a liquid dosage form or solid dosage form that is not covalently bound to a molecule or chemical substance. In some embodiments, the compositions, pharmaceutical compositions or decontamination solution of the disclosure comprises a non-bonded amino acid salt which may be complexed with a buffer, salt or other small chemical compound, but the amino acid is not integrated within a polypeptide. In some embodiments, the composition, pharmaceutical composition or decontamination solution of the present disclosure comprises one or more amino acids that are bound to a chemical group or substituent that when administered to a surface or a subject and exposed to a pharmacologically active substance (environmentally available or physiologically available in a subject) is cleaved to form a free amino acid not covalently bound to a component of the composition, pharmaceutical composition or decontamination solution. This form would be considered a non-toxic pro-drug form of the amino acid. “Non-bonded” forms of the claimed amino acids include pro-drug forms that may or may not have a cleavable substituent that, under therapeutically effective conditions, cleaved from the amino acid or amino acids in the composition.

The compositions of the present invention may be used to treat an animal (a subject) having a biofilm-producing microorganism that is causing an infection. The compositions of the present invention may also be used in the prevention of this type of infection. In some embodiments, the subject may be a human patient or a mammal to whom the compositions of the present invention may be provided or administered. In some embodiments, the subject may be a non-mammalian animal to whom the present invention is provided or administered.

The pharmaceutical compositions of the present invention may include a therapeutically effective amount of one or more non-bonded amino acids, which are effective to reduce signs and symptoms associated with any bacterial, fungal, hybrid, or protozoan infections, as determined by any means suitable in the art. Such results may include, but are not limited to, the effective disruption of bacterial biofilm growth or maintenance, the effective disruption of fungal biofilm growth or maintenance, the effective disruption of protozoan biofilm growth or maintenance, and/or the reduction of clinically relevant numbers of bacterial, fungal, or protozoan cells at or proximate to the surface of an implanted or non-implanted medical device or surface intended to be sterile. The effective amount of the composition may be dependent on any number of variables, including without limitation, the species, breed, size, height, weight, age, overall health of the subject, the type of formulation, the mode or manner or administration, the type and/or severity of the particular condition being treated.

An object of the disclosure is a pharmaceutical composition comprising one or more non-bonded amino acids in a therapeutically effective amount. In some embodiments, the non-bonded amino acid(s) of the composition may be in a liquid form dissolved at a particular concentration. In some embodiments, a pharmaceutical composition or formulation disclosed herein may be formulated for administration intravenously, intra-arterially, orally, gastrointestinally, through irrigation of a wound, intradermaly, intramucosally, subcutaneously, intramuscularly, intracavernously, intraocularly, intranasally, into a sinus, intraductally, intrathecally, subdurally, extradurally, intraventricular, into the plural space, into an abscess, into an infected cyst, into an infected tissue, into a myofascial plane, intraarticularly, intrauterinely, or into the peritoneal cavity, and may comprise one or more of the disclosed non-bonded amino acids in a total amino acid concentration of from about 0.01% to about 30.0% (e.g., about 0.01% to about 20.0%, about 0.01% to about 10.0%, and other ranges) in weight to volume of solution. In some embodiments, the non-bonded amino acids of any of the compositions or pharmaceutical compositions disclosed here may have a concentration of from about 0.01% to about 29% weight to volume, from about 0.01% to about 28% weight to volume, from about 0.01% to about 27% weight to volume, from about 0.01% to about 26% weight to volume, from about 0.01% to about 25% weight to volume, from about 0.01% to about 24% weight to volume, from about 0.01% to about 23% weight to volume, from about 0.01% to about 22% weight to volume, from about 0.01% to about 21% weight to volume, from about 0.01% to about 20% weight to volume, from about 0.01% to about 19% weight to volume, from about 0.01% to about 18% weight to volume, from about 0.01% to about 17% weight to volume, from about 0.01% to about 16% weight to volume, from about 0.01% to about 15% weight to volume, from about 0.01% to about 14% weight to volume, from about 0.01% to about 13% weight to volume, from about 0.01% to about 12% weight to volume, from about 0.01% to about 11% weight to volume, from about 0.01% to about 10% weight to volume, from about 0.01% to about 9% weight to volume, from about 0.01% to about 8% weight to volume, from about 0.01% to about 7% weight to volume, from about 0.01% to about 6% weight to volume, from about 0.01% to about 5% weight to volume, from about 0.01% to about 4% weight to volume, from about 0.01% to about 3% weight to volume, from about 0.01% to about 2% weight to volume, from about 0.01% to about 1% weight to volume, from about 0.01% to about 0.5% weight to volume, from about 0.01% to about 0.25% weight to volume, from about 0.01% to about 0.1% weight to volume, from about 0.1% to about 5% weight to volume, from about 0.1% to about 3% weight to volume, from about 0.1% to about 4% weight to volume, from about 0.1% to about 3% weight to volume, from about 0.1% to about 2% weight to volume, from about 0.1% to about 1% weight to volume, from about 0.1% to about 0.5% weight to volume, from about 1% to about 5% weight to volume, from about 1% to about 4% weight to volume, from about 1% to about 3% weight to volume, from about 1% to about 2% weight to volume, from about 0.5% to about 3% weight to volume, from about 0.5% to about 2.5% weight to volume, from about 0.5% to about 2% weight to volume, from about 0.5% to about 1.5% weight to volume, from about 0.5% to about 1% weight to volume, or any other value or range of values therein. Such pharmaceutical compositions or formulations may include one or more of the following non-bonded amino acids: aspartic acid, or pharmaceutically acceptable salts thereof; cysteine, or pharmaceutically acceptable salts thereof; glutamic acid, or pharmaceutically acceptable salts thereof or also the following amino acids or pharmaceutically acceptable salts thereof, including beta alanine or pharmaceutically acceptable salts thereof, 2-aminoadipic acid or pharmaceutically acceptable salts thereof, cystathionine or pharmaceutically acceptable salts thereof, ethanolamine or pharmaceutically acceptable salts thereof, homocysteine or pharmaceutically acceptable salts thereof, hydroxyproline or pharmaceutically acceptable salts thereof, phosphoethanolamine or pharmaceutically acceptable salts thereof, or phosphoserine or pharmaceutically acceptable salts thereof.

In some embodiments, the pharmaceutical composition or formulation disclosed herein may be formulated for administration gastrointestinally, topically, via a wound dressing, sublingually, orally, intravaginally, intrarectally, transmucosally, transdermally, intrapulmonary, on infected skin, burns, on a cutaneous ulcer, on infected nails, by swish and swallow treatment of oral candidiasis, or otherwise using a cream, ointment, gel, or other topical medicament may comprise one or more of the disclosed non-bonded amino acids in a total amino acid concentration range of about 0.1% to approximately 100% in weight to volume of solid solution. In some embodiments, the amino acids of any of the pharmaceutical compositions disclosed here may have a dosage of from about 0.1% to about 95% weight to volume, a dosage of from about 0.1% to about 85% weight to volume, a dosage of from about 0.1% to about 80% weight to volume, a dosage of from about 0.1% to about 75% weight to volume, from about 0.1% to about 70% weight to volume, from about 0.1% to about 65% weight to volume, from about 0.1% to about 60% weight to volume, from about 0.1% to about 55% weight to volume, from about 0.1% to about 50% weight to volume, from about 0.1% to about 45% weight to volume, from about 0.1% to about 40% weight to volume, from about 0.1% to about 35% weight to volume, from about 0. 1% to about 30% weight to volume, from about 0.1% to about 25% weight to volume, from about 0.1% to about 20% weight to volume, from about 0.1% to about 15% weight to volume, from about 0.1% to about 10% weight to volume, from about 0.1% to about 5% weight to volume, from about 0.1% to about 2.5% weight to volume, from about 0.1% to about 1.5% weight to volume, from about 0.1% to about 1% weight to volume, from about 5% to about 80% weight to volume, from about 5% to about 75% weight to volume, from about 5% to about 70% weight to volume, from about 5% to about 65% weight to volume, from about 5% to about 60% weight to volume, from about 5% to about 55% weight to volume, from about 5% to about 50% weight to volume, from about 5% to about 45% weight to volume, from about 5% to about 40% weight to volume, from about 5% to about 35% weight to volume, from about 5% to about 30% weight to volume, from about 5% to about 25% weight to volume, from about 5% to about 20% weight to volume, from about 5% to about 15% weight to volume, from about 5% to about 10% weight to volume, or any other value or range of values therein. Such pharmaceutical composition or formulation may include one or more of the following non-bonded amino acids: aspartic acid, or pharmaceutically acceptable salts thereof; cysteine, or pharmaceutically acceptable salts thereof; glutamic acid, or pharmaceutically acceptable salts thereof beta alanine or pharmaceutically acceptable salts thereof, 2-aminoadipic acid or pharmaceutically acceptable salts thereof, cystathionine or pharmaceutically acceptable salts thereof, ethanolamine or pharmaceutically acceptable salts thereof, homocysteine or pharmaceutically acceptable salts thereof, hydroxyproline or pharmaceutically acceptable salts thereof, phosphoethanolamine or pharmaceutically acceptable salts thereof, phosphoserine or pharmaceutically acceptable salts thereof.

In some embodiments, the composition or formulation disclosed herein may be formulated for disinfecting surfaces or other sterilization uses, and may comprise one or more of the disclosed non-bonded amino acids in a total amino acid concentration range from about 0.1% to approximately 100% in weight to volume of solution, 0.1% to approximately 95% weight to volume solution, 0.1% to approximately 90% weight to volume of solution, 0.1% to approximately 85% weight to volume of solution, 0.1% to approximately 80% in weight to volume of solution. In some embodiments, the amino acids of any of the pharmaceutical compositions disclosed here may have a dosage of from about 0.1% to about 75% weight to volume, from about 0.1% to about 70% weight to volume, from about 0.1% to about 65% weight to volume, from about 0.1% to about 60% weight to volume, from about 0.1% to about 55% weight to volume, from about 0.1% to about 50% weight to volume, from about 0.1% to about 45% weight to volume, from about 0.1% to about 40% weight to volume, from about 0.1% to about 35% weight to volume, from about 0. 1% to about 30% weight to volume, from about 0.1% to about 25% weight to volume, from about 0.1% to about 20% weight to volume, from about 0.1% to about 15% weight to volume, from about 0.1% to about 10% weight to volume, from about 0.1% to about 5% weight to volume, from about 0.1% to about 2.5% weight to volume, from about 0.1% to about 1.5% weight to volume, from about 0.1% to about 1% weight to volume, from about 5% to about 80% weight to volume, from about 5% to about 75% weight to volume, from about 5% to about 70% weight to volume, from about 5% to about 65% weight to volume, from about 5% to about 60% weight to volume, from about 5% to about 55% weight to volume, from about 5% to about 50% weight to volume, from about 5% to about 45% weight to volume, from about 5% to about 40% weight to volume, from about 5% to about 35% weight to volume, from about 5% to about 30% weight to volume, from about 5% to about 25% weight to volume, from about 5% to about 20% weight to volume, from about 5% to about 15% weight to volume, from about 5% to about 10% weight to volume, or any other value or range of values therein. Such compositions or formulation may include one or more of the following non-bonded amino acids: aspartic acid, or acceptable salts thereof; cysteine, or acceptable salts thereof; glutamic acid, or acceptable salts thereof; beta alanine or acceptable salts thereof, 2-aminoadipic acid or acceptable salts thereof, cystathionine or acceptable salts thereof, ethanolamine or acceptable salts thereof, homocysteine or acceptable salts thereof, hydroxyproline or acceptable salts thereof, phosphoethanolamine or acceptable salts thereof, or phosphoserine or acceptable salts thereof.

In some embodiments, the composition or formulation disclosed herein may be formulated as a concentrate for later dilution, and may comprise one or more of the disclosed non-bonded amino acids in a total amino acid concentration range from about 1% to about 100% in by weight of a solid solution. The present invention includes pharmaceutical concentrates that may be used for inclusion in a medicament, such as an injectable solution, wound irrigation solution, gels or pastes for wound dressings, etc. The present invention further includes concentrates for creating disinfection solutions and compositions. In some embodiments, the amino acids of any of the concentrate compositions disclosed here may have a dosage of from about 1% to about 100% weight. In some embodiments, the concentrate compositions disclosed here may have a dosage of from about 1% to about 95% weight, from about 1% to about 90% weight, from about 1% to about 85% weight, from about 1% to about 80% weight, from about 1% to about 75% weight, from about 1% to about 70% weight, from about 1% to about 65% weight, from about 1% to about 60% weight, from about 1% to about 55% weight, from about 1% to about 50% weight, from about 1% to about 45% weight, from about 1% to about 40% weight, from about 1% to about 35% weight, from about 1% to about 30% weight, from about 1% to about 25% weight, from about 1% to about 20% weight, from about 1% to about 15% weight, from about 1% to about 10% weight, from about 1% to about 5% weight, from about 0.1% to about 2.5% weight, from about 0.1% to about 1.5% weight, from about 0.1% to about 1% weight, from about 5% to about 80% weight, from about 5% to about 75% weight, from about 5% to about 70% weight, from about 5% to about 65% weight, from about 5% to about 60% weight, from about 5% to about 55% weight, from about 5% to about 50% weight, from about 5% to about 45% weight, from about 5% to about 40% weight, from about 5% to about 35% weight, from about 5% to about 30% weight, from about 5% to about 25% weight, from about 5% to about 20% weight, from about 5% to about 15% weight, from about 5% to about 10% weight, or any other value or range of values therein. Such pharmaceutical composition or formulation may include one or more of the following non-bonded amino acids: aspartic acid, pharmaceutically acceptable salts thereof, or other salts thereof; cysteine, pharmaceutically acceptable salts thereof, or other salts thereof; glutamic acid, pharmaceutically acceptable salts thereof, or other salts thereof beta alanine or pharmaceutically acceptable salts thereof, 2-aminoadipic acid or pharmaceutically acceptable salts thereof, cystathionine or pharmaceutically acceptable salts thereof, ethanolamine or pharmaceutically acceptable salts thereof, homocysteine or pharmaceutically acceptable salts thereof, hydroxyproline or pharmaceutically acceptable salts thereof, phosphoethanolamine or pharmaceutically acceptable salts thereof, phosphoserine or pharmaceutically acceptable salts thereof.

In some embodiments, the invention relates to a pharmaceutical composition used to prevent a bacterial infection from developing a biofilm. In such embodiments, the pharmaceutical composition may comprise one or more of the following non-bonded amino acids: aspartic acid, pharmaceutically acceptable salts thereof, or other salts thereof; cysteine, pharmaceutically acceptable salts thereof, or other salts thereof; glutamic acid, pharmaceutically acceptable salts thereof, or other salts thereof; beta alanine or pharmaceutically acceptable salts thereof, 2-aminoadipic acid or pharmaceutically acceptable salts thereof, cystathionine or pharmaceutically acceptable salts thereof, ethanolamine or pharmaceutically acceptable salts thereof, homocysteine or pharmaceutically acceptable salts thereof, hydroxyproline or pharmaceutically acceptable salts thereof, phosphoethanolamine or pharmaceutically acceptable salts thereof, or phosphoserine or pharmaceutically acceptable salts thereof. Such pharmaceutical compositions may be prepared with one or more of the foregoing amino acids in an appropriate total amino acid concentration range, as described above (e.g., an injectable liquid solution in a total amino acid concentration range of about 0.01% to about 30.0% weight to volume).

In some embodiments, the invention relates to a pharmaceutical composition used treat a bacterial infection that has developed a biofilm. In such embodiments, the pharmaceutical composition may comprise one or more of the following non-bonded amino acids: aspartic acid, pharmaceutically acceptable salts thereof, or other salts thereof; cysteine, pharmaceutically acceptable salts thereof, or other salts thereof; glutamic acid, pharmaceutically acceptable salts thereof, or other salts thereof; beta alanine or pharmaceutically acceptable salts thereof, 2-aminoadipic acid or pharmaceutically acceptable salts thereof, cystathionine or pharmaceutically acceptable salts thereof, ethanolamine or pharmaceutically acceptable salts thereof, homocysteine or pharmaceutically acceptable salts thereof, hydroxyproline or pharmaceutically acceptable salts thereof, phosphoethanolamine or pharmaceutically acceptable salts thereof, or phosphoserine or pharmaceutically acceptable salts thereof. Such pharmaceutical compositions may be prepared with one or more of the foregoing amino acids in an appropriate total amino acid concentration range, as described above (e.g., an injectable liquid solution in a total amino acid concentration range of about 0.01% to about 30.0% weight to volume).

In some embodiments, the invention relates to a pharmaceutical composition used to prevent a fungal infection from developing a biofilm. In such embodiments, the pharmaceutical composition may comprise one or more of the following non-bonded amino acids: aspartic acid, pharmaceutically acceptable salts thereof, or other salts thereof; cysteine, pharmaceutically acceptable salts thereof, or other salts thereof; glutamic acid, pharmaceutically acceptable salts thereof, or other salts thereof; beta alanine or pharmaceutically acceptable salts thereof, 2-aminoadipic acid or pharmaceutically acceptable salts thereof, cystathionine or pharmaceutically acceptable salts thereof, ethanolamine or pharmaceutically acceptable salts thereof, homocysteine or pharmaceutically acceptable salts thereof, hydroxyproline or pharmaceutically acceptable salts thereof, phosphoethanolamine or pharmaceutically acceptable salts thereof, or phosphoserine or pharmaceutically acceptable salts thereof. Such pharmaceutical compositions may be prepared with one or more of the foregoing amino acids in an appropriate total amino acid concentration range, as described above (e.g., an injectable liquid solution in a total amino acid concentration range of about 0.01% to about 30.0% weight to volume).

In some embodiments, the invention relates to a pharmaceutical composition used to treat a fungal infection that has developed a biofilm. In such embodiments, the pharmaceutical composition may comprise one or more of the following non-bonded amino acids: aspartic acid, pharmaceutically acceptable salts thereof, or other salts thereof; cysteine, pharmaceutically acceptable salts thereof, or other salts thereof; glutamic acid, pharmaceutically acceptable salts thereof, or other salts thereof; beta alanine or pharmaceutically acceptable salts thereof, 2-aminoadipic acid or pharmaceutically acceptable salts thereof, cystathionine or pharmaceutically acceptable salts thereof, ethanolamine or pharmaceutically acceptable salts thereof, homocysteine or pharmaceutically acceptable salts thereof, hydroxyproline or pharmaceutically acceptable salts thereof, phosphoethanolamine or pharmaceutically acceptable salts thereof, or phosphoserine or pharmaceutically acceptable salts thereof. Such pharmaceutical compositions may be prepared with one or more of the foregoing amino acids in an appropriate total amino acid concentration range, as described above (e.g., an injectable liquid solution in a total amino acid concentration range of about 0.01% to about 30.0% weight to volume).

In some embodiments, the invention relates to a pharmaceutical composition used treat or prevent a bacterial, fungal, protozoan, or a hybrid infection that has developed or may develop a biofilm. In such embodiments, the pharmaceutical composition may comprise one or more of the following non-bonded amino acids: aspartic acid, pharmaceutically acceptable salts thereof, or other salts thereof; cysteine, pharmaceutically acceptable salts thereof, or other salts thereof; glutamic acid, pharmaceutically acceptable salts thereof, or other salts thereof; beta alanine or pharmaceutically acceptable salts thereof, 2-aminoadipic acid or pharmaceutically acceptable salts thereof, cystathionine or pharmaceutically acceptable salts thereof, ethanolamine or pharmaceutically acceptable salts thereof, homocysteine or pharmaceutically acceptable salts thereof, hydroxyproline or pharmaceutically acceptable salts thereof, phosphoethanolamine or pharmaceutically acceptable salts thereof, or phosphoserine or pharmaceutically acceptable salts thereof. Such pharmaceutical compositions may be prepared with one or more of the foregoing amino acids in an appropriate total amino acid concentration range, as described above (e.g., an injectable liquid solution in a total amino acid concentration range of about 0.01% to about 30.0% weight to volume or a solid material that may contain a total amino acid concentration range of about 0.01% to about 100% weight to volume).

In some embodiments, the invention relates to a disinfecting composition used to disinfect surfaces, such as lab benches and equipment, medical and surgical tools and equipment, food preparation surfaces, food stuff, tools, and equipment, and other surfaces and structures that require disinfection, including water systems. In such embodiments, the disinfecting composition may comprise one or more of the following non-bonded amino acids: aspartic acid, pharmaceutically acceptable salts thereof, or other salts thereof; cysteine, pharmaceutically acceptable salts thereof, or other salts thereof; glutamic acid, pharmaceutically acceptable salts thereof, or other salts thereof; beta alanine or pharmaceutically acceptable salts thereof, 2-aminoadipic acid or pharmaceutically acceptable salts thereof, cystathionine or pharmaceutically acceptable salts thereof, ethanolamine or pharmaceutically acceptable salts thereof, homocysteine or pharmaceutically acceptable salts thereof, hydroxyproline or pharmaceutically acceptable salts thereof, phosphoethanolamine or pharmaceutically acceptable salts thereof, or phosphoserine or pharmaceutically acceptable salts thereof. Such disinfecting compositions may be prepared with one or more of the foregoing amino acids in an appropriate total amino acid concentration range, as described above (e.g., a disinfecting solution with a total amino acid concentration range from about 0.1% to approximately 100% in weight to volume).

The dose of the composition or pharmaceutical compositions may vary. In some embodiments, the dose of the composition may be once per day or only a total of once. In some embodiments, multiple doses may be administered to the subject daily. In some embodiments, the total dosage is administered in at least two application periods. In some embodiments, the period can be an hour, a day, a month, a year, a week, or a two-week period. In an additional embodiment of the invention, the total dosage is administered in two or more separate application periods, or separate doses. In some embodiments, the methods of administering the pharmaceutical compositions of the disclosure comprise application or administration periods of once an hour, once every two hours, once every 6 hours, once every 12 hours or once a day. In some embodiments, the methods of administering the pharmaceutical compositions of the disclosure comprise application or administration periods of twice an hour or more frequently depending upon the severity of the infection of contamination or to prevent toxic side-effects from destruction of the pathogen.

In some embodiments, subjects can be administered the composition in which the composition is provided in a daily dose range of about 0.0001 mg/kg to about 5000 mg/kg of the weight of the subject. The dose administered to the subject can also be measured in terms of total amount of amino acid administered per day. In some embodiments, a subject is administered from about 0.001 to about 3000 milligrams of amino acid per day (e.g., up to about 2000 milligrams of amino acid per day, up to about 1800 milligrams of amino acid per day, up to about 1600 milligrams of amino acid per day, up to about 1400 milligrams of amino acid per day, up to about 1200 milligrams of amino acid per day, up to about 1000 milligrams of amino acid per day, up to about 800 milligrams of amino acid per day). In some embodiments, a subject is administered from about 0.001 milligrams to about 700 milligrams of amino acid per dose (e.g., up to about 650 milligrams of amino acid per dose, up to about 600 milligrams of amino acid per dose, up to about 500 milligrams of amino acid per dose, up to about 400 milligrams of amino acid per dose, up to about 300 milligrams of amino acid per dose, up to about 200 milligrams of amino acid per dose, up to about 100 milligrams of amino acid per day, up to about 50 milligrams of amino acid per dose, up to about 10 milligrams of amino acid or amino acid composition or pharmaceutically salt thereof per dose, up to about 5 milligrams of amino acid or amino acid composition or pharmaceutically salt thereof per dose, up to about 1 milligram of Amino acid or amino acid composition or pharmaceutically salt thereof per dose, up to about 0.1 milligrams of amino acid or amino acid composition or pharmaceutically salt thereof per dose, up to about 0.001 milligrams of amino acid or amino acid composition or pharmaceutically salt thereof per dose.).

In some embodiments, subjects can be administered the composition in which the composition comprising an amino acid or pharmaceutically acceptable salt thereof is administered in a daily dose range of about 0.0001 mg/kg to about 500 mg/kg of the weight of the subject (e.g., up about 450 mg/kg of the weight of the subject, up about 400 mg/kg of the weight of the subject, up about 350 mg/kg of the weight of the subject, up about 300 mg/kg of the weight of the subject, up about 250 mg/kg of the weight of the subject, up about 200 mg/kg of the weight of the subject, up about 150 mg/kg of the weight of the subject, up about 100 mg/kg of the weight of the subject, up about 50 mg/kg of the weight of the subject, up about 25 mg/kg of the weight of the subject, up about 10 mg/kg of the weight of the subject, up about 5 mg/kg of the weight of the subject, up about 1 mg/kg of the weight of the subject, up about 0.1 mg/kg of the weight of the subject, up about 0.01 mg/kg of the weight of the subject, up about 0.001 mg/kg of the weight of the subject.

The methods of the present invention include administering compositions comprising one or a plurality of amino acids disclosed herein to a subject having a bacterial, fungal, or protozoan infections. In some embodiments, the presently disclosed invention also relates to administering one or a plurality of the compositions of amino acids of the disclosure in conjunction with one or more antibiotics, such as a β-lactam antibiotic. When one or a plurality of the compositions or amino acids of the disclosure are administered in conjunction with an antibiotic, one or a plurality of non-bonded amino acids and the antibiotic can be administered simultaneously in the same composition, simultaneously in different dosage forms or sequentially, or at different times. When the one or a plurality of non-bonded amino acids and the antibiotic are administered at the same time, they can be administered as a single composition or pharmaceutical composition or they can be administered as separate pharmaceutical compositions. It is understood that when one or a plurality of non-bonded amino acids are administered in conjunction with an antibiotic, the active agents can be administered in a single combination or in multiple combinations. For example, when administered intravenously, one or a plurality of the compositions of non-bonded amino acids can be dissolved or suspended in any of the commonly used intravenous fluids and administered by infusion, and then an antibiotic can be dissolved or suspended in any of the commonly used intravenous fluids and administered by infusion. Conversely, the antibiotic can be dissolved or suspended in any of the commonly used intravenous fluids and administered by infusion, and then one or a plurality of compositions of amino acids of the disclosure can be dissolved or suspended in any of the commonly used intravenous fluids and administered by infusion. Alternatively, a pharmaceutical composition comprising one or a plurality of the non-bonded amino acids in the disclosure and an antibiotic can be dissolved or suspended together in any of the commonly used intravenous fluids and administered by infusion.

In some embodiments, the non-bonded amino acid(s) or pharmaceutically acceptable salt(s) thereof are administered with an antibiotic agent. In some embodiments, a cephalosporin antibiotic may be included in the pharmaceutical composition. In some embodiments, a carbapenen antibiotic may be included in the pharmaceutical composition. In some embodiments, a monobactam antibiotic may be included in the pharmaceutical composition. In some embodiments, a penem antibiotic may be included in the pharmaceutical composition. In some embodiments, a penicillin antibiotic may be included in the pharmaceutical composition. In some embodiments, a macrolide antibiotic may be included in the pharmaceutical composition.

In some embodiments, the non-bonded amino acid(s) or pharmaceutically acceptable salt(s) thereof are administered with an antifungal agent. In some embodiments, the antifungal agent may be a polyene (for instance, amphotericin B), an azole (for instance, fluconazole), an echinocandin (for instance, caspofungin), a nucleoside analog (for instance, 5-fluorocytosine), an allylamine (for instance, naftifine, terbinafine, or butenafine), or other antifungal agents (for instance, ciclopirox). Additional antifungals include agents that block NA synthesis including, e.g., flucytosine, and those that disrupt microtubule function including, e.g., griseofulvin. Suitable antifungals can include one of candicidin, filipin, hamycin, natamycin, and rimocidin. Triazoles, including albaconazole, fluconazole, isavuconazole, itraconazole, posaconazole, ravuconazole, terconazole, and voriconazole are also suitable antifungal active agents. Additional antifungals may include thiazoles, amorolfine, benzoic acid, haloprogin, tolnaftate, undecylenic acid, and Crystal violet. Suitable salts of antifungal agents include but are not limited to hydrochloride salts. In some embodiments, an antifungal agent is selected from the group consisting of naftifine, butenafine, terbinafine, and amorolfine and pharmaceutically acceptable salts thereof.

Methods for making the presently described antifungal agents and pharmaceutically acceptable salts thereof are disclosed in U.S. Pat. Nos. 4,755,534; 4,680,291; 4,282,251; and a published paper in the A.J. of Infectious Disease and Microbiology, 2014, Vol. 2, No. 5, 122-130, each of which is incorporated by reference herein in its entirety.

The pharmaceutical compositions can comprise one or more of the compounds disclosed herein (e.g. one or more amino acid compositions), optionally further comprising one or more nontoxic, pharmaceutically-acceptable carriers and/or diluents and/or adjuvants and/or excipients. As used herein, the phrase “pharmaceutically acceptable carrier” refers to any and all solvents, dispersion media, coatings, antibacterial and antifungal agents, isotonic and absorption delaying agents, and the like, that are compatible with pharmaceutical administration. The use of such media and agents for pharmaceutically active substances is well known in the art. Non-limiting examples of carriers and excipients include corn starch or gelatin, lactose, sucrose, microcrystalline cellulose, kaolin, mannitol, dicalcium phosphate, sodium chloride and alginic acid. The compositions may contain croscarmellose sodium, microcrystalline cellulose, corn starch, sodium starch glycolate and alginic acid.

In general, the pharmaceutical compositions of the instant disclosure or the pharmaceutical acceptable salts derived therefrom may be in a liquid or solid dosage form. Such dosage forms, may be tablets, capsules, powders, liquid formulations, delayed or sustained release, patches, snuffs, nasal sprays and the like. The formulations may additionally include other ingredients such as dyes, preservatives, buffers and anti-oxidants, for example. The physical form and content of the pharmaceutical formulations contemplated are conventional preparations that can be formulated by those skilled in the pharmaceutical formulation field.

For intravenous (IV) use, the pharmaceutical compositions, optionally in conjunction with an antibiotic or an antifungal agent, can be dissolved or suspended in any of the commonly used intravenous fluids and administered by infusion. Intravenous fluids include, without limitation, physiological saline or Ringer's solution. Intravenous administration may be accomplished by using, without limitation, syringe, mini-pump or intravenous line. Pharmaceutical compositions of this disclosure may also be formulated for parenteral injection, and include aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, benzyl alcohol, polyols (such as glycerol, propylene glycol, and polyethylene glycol), and suitable mixtures thereof, vegetable oils (such as corn oil or olive oil), and injectable organic esters such as ethyl oleate. Proper fluidity can be maintained, for example, by the use of coating materials such as lecithin, by the maintenance of the required particle size in the case of dispersions, and by the use of surfactants. The compositions can include various buffers. These compositions may also contain adjuvants such as preservatives, wetting agents, emulsifying agents, and dispersing agents. They may also contain taggants or other anti-counterfeiting agents, which are well known in the art. Prevention of the action of microorganisms may be ensured by the inclusion of various antibacterial and antifungal agents, for example, paraben, chlorobutanol, and phenol sorbic acid. It may also be desirable to include isotonic agents such as sugars and sodium chloride. Prolonged absorption of the injectable pharmaceutical form may be brought about by the inclusion of agents which delay absorption, such as aluminum monostearate and gelatin. The injectable formulations can be sterilized, for example, by filtration through a bacterial-retaining filter, or by incorporating sterilizing agents in the form of sterile solid compositions, which can be dissolved or dispersed in sterile water or other sterile injectable medium just prior to use or storage.

Injectable depot forms can be made by forming microencapsulating matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations can also be prepared by entrapping the drug in liposomes or microemulsions, which are compatible with body tissues.

For example, the pharmaceutical compositions of the present invention may be administered as a dose of an intravenous, intra-arterially, orally, gastrointestinally, intramuscular, or parenteral formulation of compounds, preferably a composition, pharmaceutical composition or formulation disclosed herein, may be administered as a bolus or by slow infusion. A bolus is a dose that is administered in less than 30 minutes. In one embodiment, a bolus is administered in less than 15 or less than 10 minutes. In another embodiment, a bolus is administered in less than 5 minutes. In yet another embodiment, a bolus is administered in one minute or less. An infusion is a dose that is administered at a rate of 30 minutes or greater. In one embodiment, the infusion is one hour or greater. In another embodiment, the infusion is substantially constant.

As a further example, the pharmaceutical compositions of the present invention may be administered topically, where the pharmaceutical composition or formulation is prepared in suitable forms to be applied to the skin, to cutaneous ulcers, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient.

As a further example, the pharmaceutical compositions of the present invention may be prepared for application to the eyes or ears, where the pharmaceutical composition can be presented in liquid or semi-liquid form formulated in hydrophobic or hydrophilic bases as ointments, creams, lotions, paints or powders.

As a further example, the pharmaceutical compositions of the present invention may be prepared for rectal and vaginal administration, where the pharmaceutical compositions are in the form of suppositories admixed with conventional carriers such as cocoa butter, polyethylene glycol or a suppository wax or other glyceride that are solid at room temperature but liquid at body temperature and therefore melt in the rectum or vaginal cavity and release the active compound. Also as further example, the pharmaceutical compositions of the present invention may be prepared for intrapulmonary administration such as an inhalation.

In general, the methods of delivering the pharmaceutical compositions in vivo utilize art-recognized protocols for delivering the agent with the only substantial procedural modification being the substitution of the compounds of the present invention for the drugs in the art-recognized protocols. Likewise, methods for using the claimed compositions for treating cells in culture, for example, to eliminate or reduce the level of bacterial contamination of a cell culture, utilize art-recognized protocols for treating cell cultures with antibacterial agent(s) with the only substantial procedural modification being the substitution of the compounds of the present invention, optionally in combination with an antibiotic for the drugs in the art-recognized protocols.

Exemplary procedures for delivering pharmaceutical compounds are also described in U.S. Pat. Nos. 6,468,967; 6,852,689; and U.S. Pat. No. 5,041,567, issued to Rogers and in PCT patent application number EP94/02552 (publication no. WO 95/05384), the disclosures of which are incorporated by reference in their entireties.

The disclosure also relates to the compositions, pharmaceutical compositions or formulations disclosed herein for use in treatment of a surface for disinfecting purposes. In some embodiments, the surface is a surface of an implantable device or a catheter or drain tube. The amino acid compositions of the present invention may be applied in sterilization of surfaces, such as countertops, walls, etc.; in sterilization of equipment such as surgical equipment, respirators, etc.; on the bodies of water and in the treatment of water delivery systems; and in the treatment of plants and trees. The amino acid compositions may be used in the sterilization of food processing plants and on/in food stuffs in order to prevent bacterial and fungal biofilm.

The disclosure also relates to the therapeutic use of compositions comprising one or a plurality of non-bonded amino acids or pharmaceutically acceptable salts thereof for treating fungal, bacterial, hybrid, or protozoan infections that produce biofilms.

In one aspect, the present invention related to a pharmaceutical composition for reducing or inhibiting bacterial biofilms, fungal biofilms, hybrid infection biofilms, and protozoan biofilms comprising at least one non-bonded non-toxic amino acid. The composition can be administered to an animal, including humans, in order to treat infection caused by biofilm producing bacteria, fungi, hybrid infections, and protozoa. The composition may further comprising glycerol, which may potentiate the at least one non-bonded, non-toxic amino acid. The composition may include non-bonded, non-toxic aspartic acid, or a pharmaceutically acceptable salt thereof. The composition may include non-bonded, non-toxic cysteine, or a pharmaceutically acceptable salt thereof. The composition may include non-bonded non-toxic glutamic acid, or a pharmaceutically acceptable salt thereof. The composition may further include at least one additional non-bonded non-toxic amino acid, or a pharmaceutically acceptable salt thereof, selected from beta-alanine, 2-aminoadipic acid, cystathionine, ethanolamine, homocysteine, hydroxyproline, phosphoethanolamine, and phosphoserine. The composition may further include at least one additional non-bonded non-toxic amino acid, or a pharmaceutically acceptable salt thereof, selected from 2-aminobutyric acid, glutamine, histidine, 1-methylhistidine, and tyrosine. The non-bonded, non-toxic amino acids, or pharmaceutically acceptable salts thereof, may be present in the composition at total amino acid concentration of 0.01% to about 100% weight to volume. The composition may further include glycerol, which may potentiate the at least one non-bonded, non-toxic amino acid. The composition may be effective to prevent biofilm formation and reduces a treated biofilm by at least 30%.

In some embodiments, the pharmaceutical composition may be a liquid, paste, gel, powder, granules, solids such as slivers, or an aerosol. In some embodiments, the pharmaceutical composition may be a concentrate that may be diluted for administration to a patient, the concentrate having a total amino acid concentration range of about 1.0% to about 100.0% weight to volume. In some embodiments, the pharmaceutical composition may be administered for treatment to a patient intravenously, intra-arterially, gastrointestinally, through irrigation of a wound, intradermaly, intramucosally, subcutaneously, intramuscularly, intracavernously, intraocularly, intranasally, into a sinus, intraductally, intrathecally, subdurally, extradurally, intraventricular, intrapulmonary, into the plural space, intraarticularly, intrauterinely, into the peritoneal cavity, the composition having a total amino acid concentration range of about 0.01% to about 30.0% weight to volume. In some embodiments, the pharmaceutical composition may be administered for treatment to a patient topically, via a wound dressing, sublingually, orally, intravaginally, intranasally, intrarectally, transmucosally, or transdermally, intrapulmonary, by swish and swallow treatment for oral candidiasis, into an abscess, or into an infected cyst, the composition having a total amino acid concentration range of about 0.1% to about 95.0% weight to volume. In some embodiments, the pharmaceutical composition may be administered by injection directly into an infected tissue or myofascial planes, the composition having a total amino acid concentration of about 0.1% to approximately 60%. In some embodiments, the pharmaceutical composition may be administered on infected skin, mucous membranes, toenails, or cutaneous ulcers, aid composition having a total amino acid concentration of about 10% to approximately 100%.

In some embodiments, the composition may include non-bonded, non-toxic aspartic acid non-bonded, non-bonded non-toxic cysteine, and non-toxic glutamic acid, or pharmaceutically acceptable salts thereof, and may prevent biofilm formation and reduce a treated biofilm by at least 90%.

In another aspect, the present invention related to a disinfecting composition comprising a solution of at least one of non-bonded non-toxic aspartic acid, or a salt thereof; non-bonded non-toxic cysteine, or a salt thereof; and non-bonded non-toxic glutamic acid, or a salt thereof, wherein the composition prevents biofilm formation and reduces a treated biofilm by at least 30%. The disinfecting composition may include non-bonded non-toxic aspartic acid, or a salt thereof; non-bonded non-toxic cysteine, or a salt thereof; and non-bonded non-toxic glutamic acid, or a salt thereof, and the disinfecting composition may prevent biofilm formation and reduce a treated biofilm by at least 90%. The disinfecting composition may further include at least one additional non-bonded non-toxic amino acid, or a salt thereof, selected from beta-alanine, 2-aminoadipic acid, cystathionine, ethanolamine, homocysteine, hydroxyproline, o-phosphoethanolamine, and phosphoserine. The disinfecting composition may further include at least one additional non-bonded non-toxic amino acid, or a pharmaceutically acceptable salt thereof, selected from 2-aminobutyric acid, glutamine, histidine, 1-methylhistidine, and tyrosine. The disinfecting composition may further include glycerol. The total amino acid concentration of the pharmaceutical composition may be from about 0.1% to about 95% weight to volume.

The disinfecting composition may be used in a sanitation method that introduces the composition into a water distribution system, a food processing environment, a food preparation surface or equipment, on or in food stuffs, on plants or parts thereof, or in other system or objects that require disinfection. The disinfecting composition may also be used in a sanitation method that applies the composition to medical equipment, such as respirators, endotracheal tubes, and other medical instruments and devices (e.g., reusable instruments and devices) that require sanitation. The disinfecting composition may also be used in a sanitation method that applies the composition to surfaces in need of sterilization including walls ceilings, and floors.

In another aspect, the present invention relates to a disinfecting composition comprising a solution of at least one of non-bonded non-toxic beta-alanine, or a salt thereof; non-bonded non-toxic 2-aminoadipic acid, or a salt thereof; non-bonded non-toxic cystathionine, or a salt thereof; non-bonded non-toxic ethanolamine, or a salt thereof; non-bonded non-toxic homocysteine, or a salt thereof; non-bonded non-toxic hydroxyproline, or a salt thereof; non-bonded non-toxic o-phosphoethanolamine, or a salt thereof; and non-bonded non-toxic phosphoserine, or a salt thereof, and the disinfecting composition prevents biofilm formation and reduces a treated biofilm by at least 10%. The disinfecting composition may further include at least one additional non-bonded non-toxic amino acid, or a pharmaceutically acceptable salt thereof, selected from 2-aminobutyric acid, glutamine, histidine, 1-methylhistidine, and tyrosine. The disinfecting composition may further include glycerol. The total amino acid concentration of the pharmaceutical composition may be from about 0.1% to about 95% weight to volume. The disinfecting composition may further include at least one of non-bonded non-toxic aspartic acid, or a salt thereof; non-bonded non-toxic cysteine, or a salt thereof; and non-bonded non-toxic glutamic acid, or a salt thereof, and the disinfecting composition may prevent biofilm formation and reduce a treated biofilm by at least 90%.

The disinfecting composition may be used in a sanitation method that introduces the composition into a water distribution system, a food processing environment, a food preparation surface or equipment, on plants or parts thereof, or in other system or objects that require disinfection. The disinfecting composition may also be used in a sanitation method that applies the composition to medical equipment, such as respirators, endotracheal tubes, and other medical instruments and devices (e.g., reusable instruments and devices) that require sanitation. The disinfecting composition may also be used in a sanitation method that applies the composition to surfaces in need of sterilization including walls ceilings, and floors.

In another aspect, the present invention relates to a pharmaceutical composition for reducing bacterial biofilm, fungal biofilm, protozoan biofilm, or hybrid infection biofilm comprising at least one non-bonded, non-toxic amino acid. The composition may include non-bonded, non-toxic aspartic acid, or a pharmaceutically acceptable salt thereof. The composition may include non-bonded cysteine, or a pharmaceutically acceptable salt thereof. The composition may include a non-bonded glutamic acid, or a pharmaceutically acceptable salt thereof. The composition may include a total amino acid concentration of about 0.01% to about 30.0% weight to volume. The composition may prevent biofilm formation and reduce a treated biofilm by at least 30%. The composition may be administered to a patient intravenously, intra-arterially, gastrointestinally, through irrigation of a wound, intradermaly, intramucosally, subcutaneously, intramuscularly, intracavernously, intraocularly, intranasally, into a sinus, intraductally, intrathecally, subdurally, extradurally, intraventricular, intrapulmonary, into the plural space, intraarticularly, intrauterinely, into the peritoneal cavity, where the composition has a total amino acid concentration range of about 0.01% to about 30.0% weight to volume. The composition may be administered to a patient topically, via a wound dressing, sublingually, orally, intravaginally, intranasally, intrarectally, transmucosally, or transdermally, intrapulmonary, by swish and swallow treatment for oral candidiasis, into an abscess, or into an infected cyst, where the composition may have a total amino acid concentration range of about 0.1% to about 95.0% weight to volume. The composition may be administered to a patient topically, via a wound dressing, sublingually, orally, intravaginally, intranasally, intrarectally, transmucosally, or transdermally, intrapulmonary, by swish and swallow treatment for oral candidiasis, into an abscess, or into an infected cyst, where the composition may have a total amino acid concentration range of about 0.1% to about 95.0% weight to volume. The composition may be administered on infected skin, mucous membranes, toenails, or cutaneous ulcers. The composition may be administered by injection directly into an infected tissue or myofascial planes. The composition may be a liquid, paste, gel, powder, granules, solids such as slivers, or an aerosol composition. The composition may be a concentrate that may be diluted for administration to a patient, the concentrate having a total amino acid concentration range of about 1.0% to about 100.0% weight to volume.

In another aspect, the present invention relates to a pharmaceutical composition for reducing or preventing bacterial, fungal, protozoan, or hybrid infection biofilm comprising a pharmaceutically acceptable carrier and at least one of non-bonded cysteine, or a pharmaceutically acceptable salt thereof; non-bonded glutamic acid, or a pharmaceutically acceptable salt thereof; and non-bonded aspartic acid, or a pharmaceutically acceptable salt thereof. The composition may include a combination of non-bonded cysteine, or a pharmaceutically acceptable salt thereof; and at least one of non-bonded glutamic acid, or a pharmaceutically acceptable salt thereof; and non-bonded aspartic acid, or a pharmaceutically acceptable salt thereof. The composition may include non-bonded cysteine, or a pharmaceutically acceptable salt thereof; and non-bonded glutamic acid, or a pharmaceutically acceptable salt thereof. The composition may include non-bonded cysteine, or a pharmaceutically acceptable salt thereof; and non-bonded aspartic acid, or a pharmaceutically acceptable salt thereof. The composition may include a combination of non-bonded glutamic acid, or a pharmaceutically acceptable salt thereof; and at least one of non-bonded cysteine, or a pharmaceutically acceptable salt thereof, and non-bonded aspartic acid, or a pharmaceutically acceptable salt thereof. The composition may include non-bonded glutamic acid, or a pharmaceutically acceptable salt thereof; and non-bonded aspartic acid, or a pharmaceutically acceptable salt thereof. The composition may include a combination of non-bonded aspartic acid, or a pharmaceutically acceptable salt thereof, and at least one of non-bonded cysteine, or a pharmaceutically acceptable salt thereof, and non-bonded glutamic acid, or a pharmaceutically acceptable salt thereof. The composition may include aspartic acid, cysteine, and glutamic acid. The composition may further include at least one additional non-bonded amino acid, or a pharmaceutically acceptable salt thereof, selected from the group consisting of beta-alanine, 2-aminoadipic acid, cystathionine, ethanolamine, homocysteine, hydroxy-proline, phosphoethanolamine, and phosphoserine. The composition may further include at least one additional non-bonded amino acid, or a pharmaceutically acceptable salt thereof, selected from the group consisting of 2-aminobutyric acid, glutamine, histidine, 1-methylhistidine, and tyrosine. The composition may further include glycerol, which potentiates the composition. The composition may prevent biofilm formation and reduce a treated biofilm by at least 50.0%. The composition may prevent biofilm formation and reduce a treated biofilm by at least 90.0%.

The composition may be a liquid, paste, gel, powder, granules, solids such as slivers, or an aerosol composition. In some embodiments, the composition may be administered to a patient intravenously, intra-arterially, gastrointestinally, through irrigation of a wound, intradermaly, intramucosally, subcutaneously, intramuscularly, intracavernously, intraocularly, intranasally, into a sinus, intraductally, intrathecally, subdurally, extradurally, intraventricular, intrapulmonary, into the plural space, intraarticularly, intrauterinely, into the peritoneal cavity, where the composition has a total amino acid concentration range of about 0.01% to about 30.0% weight to volume. In some embodiments, the composition may be administered to a patient topically, via a wound dressing, sublingually, orally, intravaginally, intranasally, intrarectally, transmucosally, or transdermally, intrapulmonary, by swish and swallow treatment for oral candidiasis, into an abscess, or into an infected cyst, where the composition has a total amino acid concentration range of about 0.1% to about 95.0% weight to volume. In some embodiments, the composition may be administered on infected skin, mucous membranes, toenails, or cutaneous ulcers. In some embodiments, the composition may be administered by injection directly into an infected tissue or myofascial planes. In some embodiments, the composition may be administered by injection directly into an infected tissue or myofascial planes.

In some embodiments, the composition may be a concentrate that may be diluted for administration to a patient, the concentrate having a total amino acid concentration range of about 1.0% to about 100.0% weight to volume.

In a further aspect, the present invention relates to a disinfecting composition comprising a solution of at least one of non-bonded cysteine, or a salt thereof; non-bonded glutamic acid, or a salt thereof; and non-bonded aspartic acid, or a salt thereof. The composition may include a combination of non-bonded cysteine, or a pharmaceutically acceptable salt thereof; and at least one of non-bonded glutamic acid, or a pharmaceutically acceptable salt thereof; and non-bonded aspartic acid, or a pharmaceutically acceptable salt thereof. The composition may include non-bonded cysteine, or a pharmaceutically acceptable salt thereof; and non-bonded glutamic acid, or a pharmaceutically acceptable salt thereof. The composition may include non-bonded cysteine, or a pharmaceutically acceptable salt thereof; and non-bonded aspartic acid, or a pharmaceutically acceptable salt thereof. The composition may include a combination of non-bonded glutamic acid, or a pharmaceutically acceptable salt thereof; and at least one of non-bonded cysteine, or a pharmaceutically acceptable salt thereof, and non-bonded aspartic acid, or a pharmaceutically acceptable salt thereof. The composition may include non-bonded glutamic acid, or a pharmaceutically acceptable salt thereof; and non-bonded aspartic acid, or a pharmaceutically acceptable salt thereof. The composition may include a combination of non-bonded aspartic acid, or a pharmaceutically acceptable salt thereof, and at least one of non-bonded cysteine, or a pharmaceutically acceptable salt thereof, and non-bonded glutamic acid, or a pharmaceutically acceptable salt thereof. The composition may include aspartic acid, cysteine, and glutamic acid. The composition may further include at least one additional non-bonded amino acid, or a pharmaceutically acceptable salt thereof, selected from the group consisting of beta-alanine, 2-aminoadipic acid, cystathionine, ethanolamine, homocysteine, hydroxy-proline, phosphoethanolamine, and phosphoserine. The composition may further include at least one additional non-bonded amino acid, or a pharmaceutically acceptable salt thereof, selected from the group consisting of 2-aminobutyric acid, glutamine, histidine, 1-methylhistidine, and tyrosine. The composition may further include glycerol. The composition may prevent biofilm formation and reduce a treated biofilm by at least 30%. The composition may have a total amino acid concentration from about 0.1% to about 95.0% weight to volume.

The composition may include aspartic acid, cysteine, and glutamic acid. The composition may prevent biofilm formation and reduce a treated biofilm by at least 50.0%. The composition may prevent biofilm formation and reduce a treated biofilm by at least 90.0%.

In a further aspect, the present invention relates to a method of treating a patient having a bacterial, fungal, protozoan, or hybrid infection that can produce a biofilm, comprising administering a pharmaceutical composition comprising at least one of glycerol and a non-bonded amino, non-toxic amino acid, or a pharmaceutically accepted salt thereof. The composition may include a combination of non-bonded cysteine, or a pharmaceutically acceptable salt thereof; and at least one of non-bonded glutamic acid, or a pharmaceutically acceptable salt thereof; and non-bonded aspartic acid, or a pharmaceutically acceptable salt thereof. The composition may include non-bonded cysteine, or a pharmaceutically acceptable salt thereof; and non-bonded glutamic acid, or a pharmaceutically acceptable salt thereof. The composition may include non-bonded cysteine, or a pharmaceutically acceptable salt thereof; and non-bonded aspartic acid, or a pharmaceutically acceptable salt thereof. The composition may include a combination of non-bonded glutamic acid, or a pharmaceutically acceptable salt thereof; and at least one of non-bonded cysteine, or a pharmaceutically acceptable salt thereof, and non-bonded aspartic acid, or a pharmaceutically acceptable salt thereof. The composition may include non-bonded glutamic acid, or a pharmaceutically acceptable salt thereof; and non-bonded aspartic acid, or a pharmaceutically acceptable salt thereof. The composition may include a combination of non-bonded aspartic acid, or a pharmaceutically acceptable salt thereof, and at least one of non-bonded cysteine, or a pharmaceutically acceptable salt thereof, and non-bonded glutamic acid, or a pharmaceutically acceptable salt thereof. The composition may include aspartic acid, cysteine, and glutamic acid. The composition may further include at least one additional non-bonded amino acid, or a pharmaceutically acceptable salt thereof, selected from the group consisting of beta-alanine, 2-aminoadipic acid, cystathionine, ethanolamine, homocysteine, hydroxy-proline, phosphoethanolamine, and phosphoserine. The composition may further include at least one additional non-bonded amino acid, or a pharmaceutically acceptable salt thereof, selected from the group consisting of 2-aminobutyric acid, glutamine, histidine, 1-methylhistidine, and tyrosine. The composition may further include glycerol and at least one non-bonded, non-toxic amino acid, wherein the glycerol potentiates the at least one non-bonded, non-toxic amino acid. The composition may include a total amino acid concentration in the composition is about 0.01% to about 30.0% weight to volume. The composition may include a total amino acid concentration in the composition is about 0.1% to about 95.0% weight to volume. The composition may prevent biofilm formation and reduce a treated biofilm by at least 30%.

The composition used in the method may be a liquid, paste, gel, powder, granules, solids such as slivers, or an aerosol composition. In some embodiments, the composition may be administered to a patient intravenously, intra-arterially, gastrointestinally, through irrigation of a wound, intradermaly, intramucosally, subcutaneously, intramuscularly, intracavernously, intraocularly, intranasally, into a sinus, intraductally, intrathecally, subdurally, extradurally, intraventricular, intrapulmonary, into the plural space, intraarticularly, intrauterinely, into the peritoneal cavity, where the composition has a total amino acid concentration range of about 0.01% to about 30.0% weight to volume. In some embodiments, the composition may be administered to a patient topically, via a wound dressing, sublingually, orally, intravaginally, intranasally, intrarectally, transmucosally, or transdermally, intrapulmonary, by swish and swallow treatment for oral candidiasis, into an abscess, or into an infected cyst, where the composition has a total amino acid concentration range of about 0.1% to about 95.0% weight to volume. In some embodiments, the composition may be administered on infected skin, mucous membranes, toenails, or cutaneous ulcers. In some embodiments, the composition may be administered by injection directly into an infected tissue or myofascial planes. In some embodiments, the composition may be administered by injection directly into an infected tissue or myofascial planes.

The composition used in the method may include aspartic acid, cysteine, and glutamic acid. The composition may prevent biofilm formation and reduce a treated biofilm by at least 50.0%. The composition may prevent biofilm formation and reduce a treated biofilm by at least 90.0%.

### EXPERIMENTAL EXAMPLES

Procalamine and Aminosyn

It was found that the infusion administered my patient, ProcalAmine®, which contained 3% glycerol and 3% amino acids which comprised the following amino acids: Isoleucine 0.21 g per 100 ml, Leucine 0.27 g per 100 ml, Lysine (as Lysine Acetate USP 0.31 g) 0.22 g per 100 ml, Methionine 0.16 g per 100 ml, Phenylalanine 0.17 g per 100 ml, Tryptophan 0.046 g per 100 ml, Valine 0.2 g per 100 ml, Alanine 0.21 g per 100 ml, Arginine 0.29 g per 100 ml, Histidine 0.085 g per 100 ml, Proline 0.34 g per 100 ml, Serine 0.18 g per 100 ml, Glycine 0.42 g per 100 ml, Threonine 0.12 g per 100 ml, Cysteine (as L-cysteine hydrochloride monohydrate less than 0.020 g) less than 0.014 g per 100 ml, both inhibited and destroyed bacterial and fungal biofilms.

It was[ found that Aminosyn 10%, another commercially available composition of amino acids for intravenous feeding was examined. Aminosyn 10% contains no L-cysteine but does in addition contain Tyrosine, Aspartic Acid, and glutamic acid did not inhibit or destroy bacterial and fungal biofilms. It was not aided by the addition of L-cysteine for the bacterial biofilms, but was aided by the addition of L-cysteine for the fungal biofilms. When L-cysteine alone was added to the second solution, it became effective only when the concentration was increased to 0.4% and had effect only against fungal biofilm when added to the solution.

Of the two compositions, Procalamine was found to be superior in reducing bacterial and fungal biofilms. Procalamine and Aminosyn have a similar composition of amino acids except that Aminosyn has three additional amino acids: Tyrosine, which has been found to have a neutral effect on biofilm, whereas the additional two amino acids aspartic acid and glutamic acid, were found to be very active against biofilm—see results below. The only other difference between the two solutions was the source of calories used in these solutions. Procalamine used 3% glycerol whereas Aminosyn used 5% glucose. While glycerol on its own was not found to have antibiofilm effect, glycerol may had an indirect effect that enables anti-biofilm amino acids to be more effective in combating biofilm in the presence of glycerol. It may also reduce the effect of amino acids that promote bacterial and fungal biofilm, such as isoleucine, leucine, lysine, methionine, phenoalanine, tryptophan, valine, alanine, arginine, proline, serine, glycine, and threonine, which are present in both solutions.

Fungal Biofilm Assays

For fungal biofilm assays, all solutions were prepared in RPMI 1640 medium. All bacterial biofilm solutions were prepared in Trypic Soy Broth (TSB), supplemented with 1% glucose (henceforth referred to as TSB-G). All subsequent procedures were performed in a manner that maintained sterility. A solution of each amino acid to be tested was prepared in weight to volume concentrations ranging from 0.1-5.0%. Amino acids were also tested in combinations. Compound solutions were homogenized with gentle agitation in the dark (4° C., 24 hours) before use.

Non-toxic amino acids were tested as follows: L-alanine, Beta-alanine, 2-aminoadipic acid, 2-aminobutyric acid, L-arginine, L-asparagine, L-aspartic acid, L-citrulline, L-cysteine, ethanolamine, L-glutamic acid, L-glycine, L-glutamine, L-histidine, L-homocysteine, L-isoleucine, L-leucine, L-lysine, L-methionine, 3-methyl-L-histidine, L-ornithine, L-phenylalanine, O-phosphoethanolamine, L-proline, Trans-4-hydroxy-L-proline, L-serine, O-phospho-L-serine, L-taurine, L-threonine, L-tryptophan, L-tyrosine, and L-valine. Two amino acids (1-methyl-L-histidine and L-cystathionine) were prepared at a highest concentration of 0.2% due to their limited solubility. All biofilm assays were performed using 384-well non-tissue culture treated polystyrene plates. The fungal species tested are as follows: Candida albicans, Candida guilliermondii Candida parapsilosis, Candida glabrata, Candida tropicalis and Candida dubliniensis. The bacterial species tested are as follows: Staphylococcus aureus (standard wild type and methicillin-resistant strain USA300), Escherichia coli, Pseudomonas aeruginosa and Staphylococcus epidermidis.

Fungal strains were streaked on Yeast Peptone Dextrose (YPD) agar plates and incubated at 30° C. for 48 hours. A single colony from each strain to be tested was inoculated into YPD broth and grown for 12 hours, at 30° C., shaking at 225 rpm. For the fungal biofilm inhibition assay, 1 μl of saturated overnight cell culture was added to either 80 μL of RPMI-1640 or RPMI-1640 supplemented with the amino acid compound solution to be tested, in a 384-well plate. The cells were allowed to adhere to the plate for 90 minutes at 37° C. with shaking (350 rpm). Loosely bound cells were washed once with phosphate buffered saline (PBS) and 80 ul of RPMI-1640, or RPMI-1640 supplemented with the amino acid compound solution was added to the plate. The plate was further incubated for 24 hours at 37° C. with shaking (350 rpm). Media was carefully aspirated and the biofilm was measured for fungal cell concentration by optical density at 600 nm. Twelve replicates were performed for each tested condition and the reported values were normalized to the control (RPMI-1640 media only).

For the fungal biofilm disruption assays, 80 μL of RPMI-1640 was added to the plate, along with 1 μL of overnight cell culture. The cells were allowed to adhere to the plate for 90 minutes at 37° C. with shaking (350 rpm). Loosely bound cells were washed once with PBS and 80 μL of RPMI-1640 was added to the plate. The plate was further incubated for 24 hours at 37° C. with shaking (350 rpm). Media was carefully aspirated from the mature biofilm and 80 μL of RPMI-1640, or RPMI-1640 supplemented with the amino acid compound solution to be tested, was gently added to the plate. The plate was further incubated for 24 hours at 37° C. with shaking (350 rpm). Media was carefully aspirated and the biofilm was measured for fungal cell concentration by optical density at 600 nm. Twelve replicates were performed for each tested condition and the reported values are normalized to the control (RPMI-1640 media only).

Bacterial Biofilm Assays

Bacterial strains were streaked on Blood Agar plates (5% sheep blood in Tryptic Soy Broth) and incubated at 37° C. for 24 hours. A single colony from each strain to be tested was inoculated in a TSB broth and grown for 12 hours, at 37° C. with shaking (225 rpm).

For the bacterial biofilm inhibition assays, 1 μL of saturated overnight cell culture was added to either 80 μL of TSB-G, or TSB-G supplemented with the amino acid compound solution to be tested, in a 384-well plate. The cells were allowed to adhere to the plate for 60 minutes at 37° C. without shaking. The media was carefully aspirated and 80 μL of TSB-G, or TSB-G supplemented with the amino acid compound solution was added to the plate. The plate was further incubated for 24 hours at 37° C. without shaking. Media was carefully aspirated and the biofilm was measured for bacterial cell concentration by optical density at 600 nm. Eight or twelve replicates were performed for each tested condition and the reported values were normalized to the control (TSB-G media only).

For the bacterial biofilm disruption assays, 80 μL of TSB-G was added to the plate, along with 1 μL of overnight cell culture. The cells were allowed to adhere to the plate for 60 minutes at 37° C. without shaking. The media was carefully aspirated and 80 μL of TSB-G was added to the plate. The plate was further incubated for 24 hours at 37° C. without shaking. Media was carefully aspirated from the mature biofilm and 80 μL of TSB-G, or TSB-G supplemented with the amino acid to be tested, was gently added to the plate. The plate was further incubated for 24 hours at 37° C. without shaking. Media was aspirated and the biofilm was measured for bacterial cell concentration by optical density at 600 nm. Twelve replicates were performed for each tested condition and the reported values were normalized to the control (TSB-G media only).

Results for Fungal and Bacterial Biofilm Assays

Weight to volume concentrations ranging from 0.1-5.0% were tested for all amino acids individually and in several combinations. The solution found to best abolish both fungal and bacterial biofilms was: 0.5% cysteine+0.5% glutamic acid+0.5% aspartic acid.

These three amino acid solutions individually have anti-biofilm effects on fungal and bacterial biofilms (these amino acids have been shown to decrease biofilm formation on average by about twofold). Combinations of these amino acids have been shown to have a synergistic effect in reducing biofilm formation. In combination, the effects of these amino acids are significantly increased with treatments compositions that include combinations of cysteine, glutamic acid, and aspartic acid. A tenfold decrease in biofilm formation has been shown in the case of treatment with compositions that include cysteine, glutamic acid, and aspartic acid. Such treatments also result in near complete removal of biofilm, a clear indication of anti-biofilm synergy between these three amino acids when applied in combination.

At concentrations lower than 0.5% of each amino acid in combination, the biofilm inhibition and disruption rates are less effective. At concentrations above 0.5%, the incremental effectiveness against biofilms appears to be attenuated (see Table A for C. albicans data and Table B for S. aureus data). The same holds true for all species of microbes tested.

TABLE A
Biofilm Inhibition and Disruption Assays for C. albicans

| | Biofilm | Biofilm |
|---|---|---|
| | Remaining | Remaining |
| | by optical | by optical |
| | density | density |
| | measure | measure |
| Condition | (Inhibition) | (Disruption) |
| RPMI Medium | 1 | 1 |
| 0.5% L-cysteine + | 0.1 +/− 0.04 | 0.03 +/− 0.03 |
| 0.5% L-glutamic acid + | | |
| 0.5% L-aspartic acid | | |
| 0.4% L-cysteine + | 0.4 +/− 0.08 | 0.3 +/− 0.05 |
| 0.4% L-glutamic acid + | | |
| 0.4% L-aspartic acid | | |
| 2% L-cysteine + | 0.2 +/− 0.06 | 0.07 +/− 0.01 |
| 2% L-glutamic acid + | | |
| 2% L-aspartic acid | | |

TABLE B
Biofilm Inhibition and Disruption Assays for S. aureus

| | Biofilm | Biofilm |
|---|---|---|
| | Remaining | Remaining |
| | by optical | by optical |
| | density | density |
| | measure | measure |
| Condition | (Inhibition) | (Disruption) |
| TSB-G Medium | 1 | 1 |
| 0.5% L-cysteine + | 0.1 +/− 0.04 | 0.2 +/− 0.07 |
| 0.5% L-glutamic acid + | | |
| 0.5% L-aspartic acid | | |
| 0.4% L-cysteine + | 0.3 +/− 0.05 | 0.5 +/− 0.04 |
| 0.4% L-glutamic acid + | | |
| 0.4% L-aspartic acid | | |
| 2% L-cysteine + | 0.2 +/− 0.09 | 0.3 +/− 0.06 |
| 2% L-glutamic acid + | | |
| 2% L-aspartic acid | | |

Table C summarizes the results of the inhibition assay, which assesses each amino acid's ability to prevent biofilm development, promote biofilm growth, and neutral effects on bacteria (S. aureus) biofilm formation and fungal (C. albicans) biofilm formation at 1% amino acid concentrations (unless specifically noted). The statistical results of Table C have a have a p≤0.001.

Table D summarizes the results of the disruption assay, which assesses each amino acid's ability to break up (disrupt) an existing mature biofilm, promote biofilm growth, and neutral effects on bacteria (S. aureus) biofilm formation and fungal (C. albicans) biofilm formation at 1% amino acid concentrations (unless specifically noted). The statistical results of Table D have a have a p≤0.001.

For both Table C and Table D, a single dot (●) signifies an effect in a range of about 10 to about 30% change, two dots (●●) signifies an effect of greater than 30% change. In Tables C and D below, B Biofilm means Bacterial Biofilm, F Biofilm means Fungal Biofilm, and Neutral means no measured effect on the treated biofilm.

TABLE C
Results of Sustained Inhibition Biofilm Assay.

| | Reduce | Reduce | Promote | Promote | | | |
|---|---|---|---|---|---|---|---|
| | B | F | B | F | Reduce | Promotes | |
| Table C | Biofilm | Biofilm | Biofilm | Biofilm | Both | Both | Neutral |
| Alanine | | | | | | | • |
| Beta-Alanine | • | | | | | | |
| 2 Aminoadipic Acid | • | | | | | | |
| 2 Aminobutyric | | | | | | | • |
| Acid | | | | | | | |
| Arginine | | | • | | | | |
| Asparagine | | | • | | | | |
| Aspartic Acid | •• | •• | | | •• | | |
| Citrulline | | | • | | | | |
| Cystathionine (0.2%) | | | | | | | • |
| Cysteine | •• | •• | | | •• | | |
| Ethanolamine | | • | | | | | |
| Glutamine | | | | | | | • |
| Glutamic Acid | •• | •• | | | •• | | |
| Glycine | | | • | | | | |
| Histidine | | | | | | | • |
| Homocysteine (0.4%) | | • | | | | | |
| Hydroxyproline | • | • | | | | | |
| Isoleucine | | | | | | | • |
| Leucine | | | • | | | | |
| Lysine | | | • | | | | |
| Methionine | | | • | | | | |
| 1-Methylhistidine | | | | | | | • |
| 3-Methylhistidine | | | | • | | | |
| Phenylalanine | | | • | | | | |
| Ornithine | | | | | | | • |
| Phosphoethanolamine | • | | | | | | |
| Phosphoserine | | | | | | | • |
| Proline | | | | | | | • |
| Serine | | | • | | | | |
| Taurine | | | • | | | | |
| Threonine | | | • | | | | |
| Tryptophan | | | • | | | | |
| Tyrosine | | | | | | | • |
| Valine | | | | • | | | |

TABLE D
Results of Disruption Biofilm Assay.

| | Reduce | Reduce | Promote | Promote | | | |
|---|---|---|---|---|---|---|---|
| | B | F | B | F | Reduce | Promotes | |
| Table D | Biofilm | Biofilm | Biofilm | Biofilm | Both | Both | Neutral |
| Alanine | | | • | | | | |
| Beta-Alanine | | | | | | | • |
| 2 Aminoadipic Acid | • | | | | | | |
| 2 Aminobutyric | | | | | | | • |
| Acid | | | | | | | |
| Arginine | | | • | | | | |
| Asparagine | | | | | | | • |
| Aspartic Acid | •• | •• | | | •• | | |
| Citrulline | | | • | | | | |
| Cystathionine (0.2%) | • | | | | | | |
| Cysteine | •• | •• | | | •• | | |
| Ethanolamine | | | | | | | • |
| Glutamine | | | | | | | • |
| Glutamic Acid | •• | •• | | | •• | | |
| Glycine | | | • | | | | |
| Histidine | | | | | | | • |
| Homocysteine (0.4%) | | • | | | | | |
| Hydroxyproline | • | | | | | | |
| Isoleucine | | | • | | | | |
| Leucine | | | • | | | | |
| Lysine | | | • | | | | |
| Methionine | | | | | | | • |
| 1-Methylhistidine | | | | | | | • |
| 3-Methylhistidine | • | | | • | | | |
| Phenylalanine | | | • | | | | |
| Ornithine | | | • | | | | |
| Phosphoethanolamine | • | | | | | | |
| Phosphoserine | • | • | | | | | |
| Proline | | | • | | | | |
| Serine | | | • | | | | |
| Taurine | | | • | | | | |
| Threonine | | | | | | | • |
| Tryptophan | | | | | | | • |
| Tyrosine | | | | | | | • |
| Valine | | | | • | | | |

The following additional amino acids (in addition to L-cysteine, L-glutamic acid, and L-aspartic acid) showed anti-biofilm properties against fungal biofilms when administered individually in at least one of the two biofilm assays tested (sustained inhibition and/or disruption): Ethanolamine, L-homocysteine, Phosphoserine, and Hydroxyproline. When administered in combination at concentrations ranging from about 0.1 to about 5.0% weight to volume of each amino acid, Ethanolamine, L-homocysteine, Phosphoserine, and L-hydroxyproline showed about a twofold reduction in the treated biofilms.

The following additional amino acids (in addition to L-cysteine, L-glutamic acid, and L-aspartic acid) also have anti-biofilm properties against bacterial biofilms when administered individually at concentrations ranging from about 0.1 to about 5.0% weight to volume in at least one of the two biofilm assays tested (sustained inhibition and/or disruption): Beta-alanine, 2-aminoadipic acid, hydroxyproline, 0-phosphoethanolamine, 0.2% cystathionine, 3-methyl-L-histidine, phosphoserine, homocysteine, and ethanolamine.

We also discovered that certain amino acids support (improve) biofilm growth. The amino acids that may support (improve) fungal biofilm growth in at least one of the two biofilm assays tested (sustained inhibition and/or disruption) are as follows: 3-methyl-L-histidine, and L-valine. Therefore, these amino acids may be harmful to an animal at risk of infection.

The amino acids that support (improve) bacterial biofilm growth based on the two biofilm assays tested (sustained inhibition and/or disruption) are as follows: L-alanine, L-arginine, L-asparagine, L-citrulline, L-glycine, L-isoleucine, L-leucine, L-lysine, L-methionine, L-phenylalanine, L-ornithine, L-proline, L-serine, L-taurine, L-threonine, and L-tryptophan. Therefore, these amino acids may be harmful to an animal at risk of infection.

The following amino acids be neutral, having no effect on bacterial or fungal biofilms based on the results of both assays (sustained inhibition and disruption): 2-aminobutyric acid, L-glutamine, L-histidine, 1-methyl-histidine, and L-tyrosine.

1. Pharmaceutical composition for reducing or inhibiting bacterial biofilms, fungal biofilms, hybrid infection biofilms, and protozoan biofilms comprising at least one non-bonded non-toxic amino acid.

2. The composition of claim 1, further comprising glycerol, wherein glycerol can potentiate said at least one non-bonded, non-toxic amino acid.

3. The composition of claim 1 or 2, wherein the composition can be administered to an animal, including humans, in order to treat infection caused by biofilm producing bacteria, fungi, hybrid infections, and protozoa.

4. The composition of claim 1, comprising non-bonded, non-toxic aspartic acid, or a pharmaceutically acceptable salt thereof.

5. The composition of claim 4, wherein said composition prevents biofilm formation and reduces a treated biofilm by at least 30%.

6. The composition of claim 1, comprising non-bonded non-toxic cysteine, or a pharmaceutically acceptable salt thereof.

7. The composition of claim 6, wherein said composition prevents biofilm formation and reduces a treated biofilm by at least 30%.

8. The composition of claim 1, comprising non-bonded non-toxic glutamic acid, or a pharmaceutically acceptable salt thereof.

9. The composition of claim 8, wherein said composition prevents biofilm formation and reduces a treated biofilm by at least 30%.

10. The composition of claim 1, wherein the composition comprises non-bonded, non-toxic aspartic acid non-bonded, non-bonded non-toxic cysteine, and non-bonded non-toxic glutamic acid, or pharmaceutically acceptable salts thereof, and prevents biofilm formation and reduces a treated biofilm by at least 90%.

11. The composition of claim 1, further comprising at least one additional non-bonded non-toxic amino acid, or a pharmaceutically acceptable salt thereof, selected from the group consisting of beta-alanine, 2-aminoadipic acid, cystathionine, ethanolamine, homocysteine, hydroxyproline, phosphoethanolamine, and phosphoserine.

12. The composition of claim 11, wherein said non-bonded, non-toxic amino acids, or pharmaceutically acceptable salts thereof, are present in said composition at total amino acid concentration of 0.01% to about 100% weight to volume.

13. (canceled)

14. The composition of claim 1, wherein said pharmaceutical composition may be a liquid, paste, gel, powder, granules, solids such as slivers, or an aerosol.

15. The composition of claim 1, wherein said pharmaceutical composition is a concentrate that may be diluted for administration to a patient, said concentrate having a total amino acid concentration range of about 1.0% to about 100.0% weight to volume.

16. The composition of claim 1 or 2, wherein said pharmaceutical composition can be administered for treatment to an animal, including humans, intravenously, intra-arterially, gastrointestinally, through irrigation of a wound, intradermaly, intramucosally, subcutaneously, intramuscularly, intracavernously, intraocularly, intranasally, into a sinus, intraductally, intrathecally, subdurally, extradurally, intraventricular, intrapulmonary, into the plural space, intraarticularly, intrauterinely, into the peritoneal cavity, said composition having a total amino acid concentration range of about 0.01% to about 30.0% weight to volume.

17. The composition of claim 1 or 2, wherein said pharmaceutical composition can be administered for treatment to an animal, including humans, topically, via a wound dressing, sublingually, orally, intravaginally, intranasally, intrarectally, transmucosally, or transdermally, intrapulmonary, by swish and swallow treatment for oral candidiasis, into an abscess, or into an infected cyst, said composition having a total amino acid concentration range of about 0.1% to about 90.0% weight to volume.

18. The composition of claim 1 or 2, wherein said pharmaceutical composition can be administered by injection directly into an infected tissue or myofascial planes, said composition having a total amino acid concentration of about 0.1% to approximately 60%.

19. The composition of claim 1 or 2, wherein said pharmaceutical composition can be administered on infected skin, mucous membranes, toenails, burns, or cutaneous ulcers, said composition having a total amino acid concentration of about 0.1% to approximately 100%.

20. The composition of claim 1 or 2, further comprising at least one additional non-bonded non-toxic amino acid, or a pharmaceutically acceptable salt thereof, selected from the group consisting of 2-aminobutyric acid, glutamine, histidine, 1-methylhistidine, and tyrosine.

21. A disinfecting composition comprising a solution of at least one of
non-bonded non-toxic aspartic acid, or a salt thereof;
non-bonded non-toxic cysteine, or a salt thereof; and
non-bonded non-toxic glutamic acid, or a salt thereof, wherein said composition prevents biofilm formation and reduces a treated biofilm by at least 30%.

22. The composition of claim 21, wherein said composition comprises
non-bonded non-toxic aspartic acid, or a salt thereof; and
non-bonded non-toxic cysteine, or a salt thereof; and
non-bonded non-toxic glutamic acid, or a salt thereof, wherein said composition reduces said treated biofilm by at least 90%.

23. The composition of claim 21, wherein the total amino acid concentration of said pharmaceutical composition is from about 0.1% to about 95% weight to volume.

24. The composition of claim 21, further comprising at least one additional non-bonded non-toxic amino acid, or a salt thereof, selected from the group consisting of beta-alanine, 2-aminoadipic acid, cystathionine, ethanolamine, homocysteine, hydroxyproline, o-phosphoethanolamine, and phosphoserine.

25. The composition of claim 24, wherein the total amino acid concentration in said composition is about 0.1% to about 95% weight to volume.

26. The composition of claim 21, further comprising glycerol.

27. A sanitation method comprising introducing the composition of claim 21 into a water distribution system, a food processing environment, a food preparation surface or equipment, foodstuffs, or on plants or parts thereof.

28. A sanitation method comprising applying the composition of claim 21 to medical equipment including respirators, endotracheal tubes, surgical equipment, and on surfaces in need of sterilization including walls ceilings, and floors.

29. (canceled)

30. A disinfecting composition comprising a solution of at least one of
non-bonded non-toxic beta-alanine, or a salt thereof;
non-bonded non-toxic 2-aminoadipic acid, or a salt thereof;
non-bonded non-toxic cystathionine, or a salt thereof;
non-bonded non-toxic ethanolamine, or a salt thereof;
non-bonded non-toxic homocysteine, or a salt thereof;
non-bonded non-toxic hydroxyproline, or a salt thereof;
non-bonded non-toxic o-phosphoethanolamine, or a salt thereof; and
non-bonded non-toxic phosphoserine, or a salt thereof;
wherein said composition prevents biofilm formation and reduces a treated biofilm by at least 10%.

31. The composition of claim 30, wherein said composition further comprises at least one of
non-bonded non-toxic aspartic acid, or a salt thereof;
non-bonded non-toxic cysteine, or a salt thereof; and
non-bonded non-toxic glutamic acid, or a salt thereof, wherein said composition reduces said treated biofilm by at least 30%.

32. The composition of claim 30, wherein the total amino acid concentration of said pharmaceutical composition is from about 0.1% to about 95% weight to volume.

33. The composition of claim 30, wherein said composition further comprises
non-bonded non-toxic aspartic acid, or a salt thereof;
non-bonded non-toxic cysteine, or a salt thereof; and
non-bonded non-toxic glutamic acid, or a salt thereof, wherein said composition reduces said treated biofilm by at least 90%.

34. The composition of claim 30, further comprising glycerol.

35. A sanitation method comprising introducing the composition of claim 30 into a water distribution system, a food processing environment, a food preparation surface or equipment, foodstuffs, or on plants or parts thereof.

36. A sanitation method comprising applying the composition of claim 30 to medical equipment including respirators, endotracheal tubes, surgical equipment, and on surfaces in need of sterilization including walls ceilings, and floors.

37. (canceled)

38-127. (canceled)
